(19) United States
(12) Patent Application Publication
(10) Pub. No.: US 2020/0334559 A1
(43) Pub. Date: Oct. 22, 2020

(54) SYSTEMS AND METHODS FOR SUPPRESSION OF INTERFERENCES IN MAGNETOENCEPHALOGRAPHY (MEG) AND OTHER MAGNETOMETER MEASUREMENTS

(71) Applicant: HI LLC, Los Angeles, CA (US)

(72) Inventors: Kristopher Anderson, Vista, CA (US); Antonio H. Lara, Sherman Oaks, CA (US); Sohrab Saeb, Santa Monica, CA (US)

(21) Appl. No.: 16/850,380

(22) Filed: Apr. 16, 2020

Related U.S. Application Data
(60) Provisional application No. 62/888,858, filed on Aug. 19, 2019.
(60) Provisional application No. 62/836,421, filed on Apr. 19, 2019.

(51) Int. Cl. G06N 7/00; G01R 33/00; G06N 20/00

(52) U.S. Cl. CPC G06N 7/005; A61B 5/04008; G06N 20/00; G01R 33/0064

(57) ABSTRACT

A magnetic field measurement system, non-transitory computer-readable medium or method can include instructions for, or performance of, actions including receiving output of multiple first magnetic field sensors and multiple second magnetic field sensors; and demixing, using the output of the first and second magnetic field sensors, at least one signal from at least one target source from signals from other magnetic field sources. The demixing may be performed using a model in which the output of the first magnetic field sensors includes the at least one signal from the at least one target source and that the output of the second magnetic field sensors does not include the at least one signal from the at least one target source.

### CROSS-REFERENCE TO RELATED APPLICATIONS

This application claims the benefit of U.S. Provisional Patent Applications Ser. Nos. 62/836,421, filed Apr. 19, 2019, and 62/888,858, filed Aug. 19, 2019, both of which are incorporated herein by reference in their entireties.

### FIELD

The present disclosure is directed to the area of magnetic field measurement systems including systems for magnetoencephalography (MEG). The present disclosure is also directed to magnetic field measurement systems and methods for suppressing background or interfering magnetic fields.

### BACKGROUND

In the nervous system, neurons propagate signals via action potentials. These are brief electric currents which flow down the length of a neuron causing chemical transmitters to be released at a synapse. The time-varying electrical currents within an ensemble of neurons generate a magnetic field. Magnetoencephalography (MEG), the measurement of magnetic fields generated by the brain, is one method for observing these neural signals.

Existing systems for observing or measuring MEG typically utilize superconducting quantum interference devices (SQUIDs) or collections of discrete optically pumped magnetometers (OPMs). SQUIDs require cryogenic cooling which is bulky and expensive and requires a lot of maintenance which preclude their use in mobile or wearable devices.

### BRIEF SUMMARY

One embodiment is a magnetic field measurement system that includes a plurality of first magnetic field sensors and a plurality of second magnetic field sensors, wherein the first and second magnetic field sensors are configured and arranged so that the first magnetic field sensors are positionable to receive at least one signal from at least target source with the first magnetic field sensors positioned closer to the at least one target source than the second magnetic field sensors; at least one memory; at least one processor coupled to the at least one memory and the first and second magnetic field sensors and configured to receive output of the first and second magnetic field sensors, wherein the at least one processor is configured to perform actions including; receiving output of the first and second magnetic field sensors; and demixing, using the output of the first and second magnetic field sensors, the at least one signal from the at least one target source from signals from other magnetic field sources.

In at least some embodiments, the first and second magnetic field sensors are disposed in a wearable article configured for placement on a head of a user. In at least some embodiments, when the wearable article is placed on the head of the user, the first magnetic field sensors are positioned closer to the head of the user than the second magnetic field sensors.

Another embodiment is a non-transitory computer-readable medium having stored thereon instructions for execution by a processor, including: receiving output of a plurality of first magnetic field sensors and a plurality of second magnetic field sensors; and demixing, using the output of the first and second magnetic field sensors, at least one signal from at least one target source from signals from other magnetic field sources, wherein the demixing is performed using a model in which the output of the first magnetic field sensors includes the at least one signal from the at least one target source and that the output of the second magnetic field sensors does not include the at least one signal from the at least one target source.

A further embodiment is a method of obtaining at least one signal from at least one target source, the method including receiving output of a plurality of first magnetic field sensors and a plurality of second magnetic field sensors; and demixing, using the output of the first and second magnetic field sensors, the at least one signal from the at least one target source from signals from other magnetic field sources, wherein the demixing is performed using a model in which the output of the first magnetic field sensors includes the at least one signal from the at least one target source and that the output of the second magnetic field sensors does not include the at least one signal from the at least one target source.

In at least some embodiments of the magnetic field measurement system, non-transitory computer-readable medium or method, the demixing is performed using a model in which the output of the first magnetic field sensors includes the at least one signal from the at least one target source and that the output of the second magnetic field sensors does not include the at least one signal from the at least one target source.

In at least some embodiments, the demixing utilizes a linear model of the signal from the at least one target source and the other magnetic field sources. In at least some embodiments, the linear model includes the following equations:

Sₙ(t)=A*Φₙ(t)+B*Φₑₓ(t)+εₙ(t)

Sₑₓ(t)=CΦₑₓ(t)+εₑₓ(t)

wherein

Sₙ(t) is a measured signal matrix from the first magnetic field sensors;

Φₙ(t) is a matrix of fields from the at least one target source;

Φₑₓ(t) is a matrix of fields from the other magnetic field sources;

εₙ(t) is a first measurement noise matrix;

Sₑₓ(t) is a measured signal matrix from the second magnetic field sensors;

εₑₓ(t) is a second measurement noise matrix;

A is a matrix that maps the at least one target source to the first magnetic field sensors;

B is a matrix that maps the other magnetic field sources to the first magnetic field sensors; and

C is a matrix that maps the other magnetic field sources to the second magnetic field sensors.

In at least some embodiments, the demixing further includes finding W, a M×N matrix from the space [symbol] ^{M×N}, that minimizes the following:

$${W}^{*} = arg {min}_{W ∈ {ℛ}^{M \times N}} {‖ {S}_{n} ( t ) - {WS}_{e x} ( t ) ‖}_{2}$$

to give

S*ₙ(t)=Sₙ(t)−W*Sₑₓ(t)

wherein

S*ₙ(t) is a signal matrix from the first magnetic field sensors with an estimate of the signals from the other magnetic field sources removed;

N is the number of first magnetic field sensors; and

M is the number of second magnetic field sensors.

In at least some embodiments, the actions or method further include adjusting W by applying S*ₙ(t) as an error term to a learning algorithm.

In at least some embodiments, the demixing further includes finding time-varying W(t), a M×N×k matrix from the space [symbol] ^{M×N×k}, that minimizes the following:

$${W}^{*} = arg {min}_{W ∈ {ℛ}^{M \times N \times k}} {‖ {S}_{n} ( t ) - {\sum }_{τ = 0}^{k - 1} W ( τ ) {S}_{e x} ( τ ) ‖}_{2}$$

to give

S*ₙ(t)=Sₙ(t)−Σ_{τ=0}^{k-1}W*(τ)Sₑₓ(t−τ)

wherein

S*ₙ(t) is a signal matrix from the first magnetic field sensors with an estimate of the signals from the other magnetic field sources removed;

N is the number of first magnetic field sensors;

M is the number of second magnetic field sensors; and

k is a number of time increments.

In at least some embodiments, the demixing utilizes a non-linear model of the signals from the at least one target source and the other magnetic field sources. In at least some embodiments, the non-linear model includes the following equations:

Sₙ(t)=A*Φₙ(t)+B*Φₑₓ(t)+εₙ(t)

Sₑₓ(t)=CΦₑₓ(t)+εₑₓ(t)

wherein

Sₙ(t) is a measured signal matrix from the first magnetic field sensors;

Φₙ(t) is a matrix of fields from the at least one target source;

Φₑₓ(t) is a matrix of fields from the other magnetic field sources;

εₙ(t) is a first measurement noise matrix;

Sₑₓ(t) is a measured signal matrix from the second magnetic field sensors;

εₑₓ(t) is a second measurement noise matrix;

A is a matrix that maps the at least one target source o the first magnetic field sensors;

B is a matrix that maps e other magnetic field sources to the first magnetic field sensors; and

C is a matrix that maps the other magnetic field sources to the second magnetic field sensors.

In at least some embodiments, the demixing further includes finding F, a non-linear function from the space [symbol] , that minimizes the following:

$${F}^{*} = arg {min}_{F ∈ ℱ} {‖ {S}_{n} - F ( {S}_{e x} ) ‖}_{2}$$

to give

S*ₙ=Sₙ−F*(Sₑₓ)

wherein

S*ₙ(t) is a signal matrix from the first magnetic field sensors with an estimate of the signals from the other magnetic field sources removed.

In at least some embodiments, the actions or method further include adjusting F by applying S*ₙ(t) as an error term to a learning algorithm.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a better understanding of the present invention, reference will be made to the following Detailed Description, which is to be read in association with the accompanying drawings, wherein:

FIG. 1A is a schematic block diagram of one embodiment of a magnetic field measurement system, according to the invention;

FIG. 1B is a schematic block diagram of one embodiment of a magnetometer, according to the invention;

FIG. 2 shows a magnetic spectrum with lines indicating dynamic ranges of magnetometers operating in different modes;

FIG. 3 is a schematic view of one embodiment of an arrangement of magnetic field sensors near a head of a user, according to the invention;

FIG. 4 is schematic illustration of calculational elements for processing sensor signals, according to the invention;

FIG. 5 is schematic illustration of one embodiment of flow utilizing a spatio-temporal linear model, according to the invention; and

FIG. 6 is a flowchart of one embodiment of a method of obtaining at least one signal from at least one target source.

### DETAILED DESCRIPTION

The present disclosure is directed to the area of magnetic field measurement systems including systems for magnetoencephalography (MEG). The present disclosure is also directed to magnetic field measurement systems and methods for suppressing background or interfering magnetic fields. Although the present disclosure utilizes magnetoencephalography (MEG) to exemplify the OPMs, systems, and methods described herein, it will be understood that the OPMs, systems, and methods can be used in any other suitable application.

Herein the terms “ambient background magnetic field” and “background magnetic field” are interchangeable and used to identify the magnetic field or fields associated with sources other than the magnetic field measurement system and the magnetic field sources of interest, such as biological source(s) (for example, neural signals from a user's brain) or non-biological source(s) of interest. The terms can include, for example, the Earth's magnetic field, as well as magnetic fields from magnets, electromagnets, electrical devices, and other signal or field generators in the environment, except for the magnetic field generator(s) that are part of the magnetic field measurement system.

The terms “gas cell”, “vapor cell”, and “vapor gas cell” are used interchangeably herein. Below, a gas cell containing alkali metal vapor is described, but it will be recognized that other gas cells can contain different gases or vapors for operation.

An optically pumped magnetometer (OPM) is a basic component used in optical magnetometry to measure magnetic fields. While there are many types of OPMs, in general magnetometers operate in two modalities: vector mode and scalar mode. In vector mode, the OPM can measure one, two, or all three vector components of the magnetic field; while in scalar mode the OPM can measure the total magnitude of the magnetic field.

Vector mode magnetometers measure a specific component of the magnetic field, such as the radial and tangential components of magnetic fields with respect the scalp of the human head. Vector mode OPMs often operate at zero-field and may utilize a spin exchange relaxation free (SERF) mode to reach femto-Tesla sensitivities. A SERF mode OPM is one example of a vector mode OPM, but other vector mode OPMs can be used at higher magnetic fields. These SERF mode magnetometers can have high sensitivity but may not function in the presence of magnetic fields higher than the linewidth of the magnetic resonance of the atoms of about 10 nT, which is much smaller than the magnetic field strength generated by the Earth. As a result, conventional SERF mode magnetometers often operate inside magnetically shielded rooms that isolate the sensor from ambient magnetic fields including Earth's magnetic field.

Magnetometers operating in the scalar mode can measure the total magnitude of the magnetic field. (Magnetometers in the vector mode can also be used for magnitude measurements.) Scalar mode OPMs often have lower sensitivity than SERF mode OPMs and are capable of operating in higher magnetic field environments.

The magnetic field measurement systems described herein can be used to measure or observe electromagnetic signals generated by one or more magnetic field sources (for example, neural signals or other biological sources) of interest. The system can measure biologically generated magnetic fields and, at least in some embodiments, can measure biologically generated magnetic fields in an unshielded or partially shielded environment. Aspects of a magnetic field measurement system will be exemplified below using magnetic signals from the brain of a user; however, biological signals from other areas of the body, as well as non-biological signals, can be measured using the system. This technology can also be applicable for uses outside biomedical sensing. In at least some embodiments, the system can be a wearable MEG system that can be used outside a magnetically shielded room. Examples of wearable MEG systems are described in U.S. Non-Provisional patent application Ser. No. 16/457,655 which is incorporated herein by reference in its entirety.

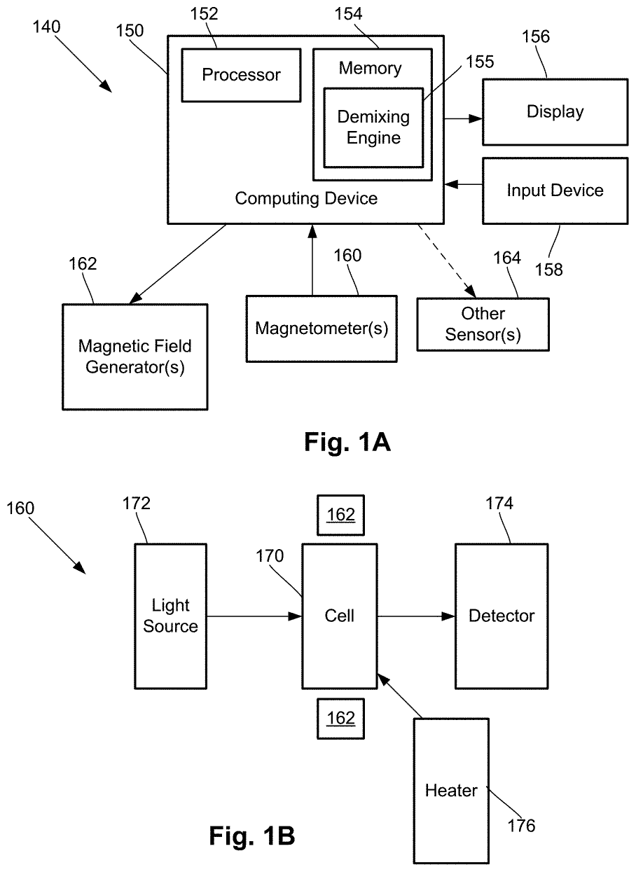

A magnetic field measurement system can utilize one or more magnetic field sensors. Magnetometers will be used herein as an example of magnetic field sensors, but other magnetic field sensors may also be used. FIG. 1A is a block diagram of components of one embodiment of a magnetic field measurement system 140. The system 140 can include a computing device 150 or any other similar device that includes a processor 152, a memory 154, a display 156, an input device 158, one or more magnetometers 160 (for example, an array of magnetometers) which can be OPMs, one or more magnetic field generators 162, and, optionally, one or more other sensors 164 (e.g., non-magnetic field sensors). The system 140 and its use and operation will be described herein with respect to the measurement of neural signals arising from one or more magnetic field sources of interest in the brain of a user as an example. It will be understood, however, that the system can be adapted and used to measure signals from other magnetic field sources of interest including, but not limited to, other neural signals, other biological signals, as well as non-biological signals.

The computing device 150 can be a computer, tablet, mobile device, field programmable gate array (FPGA), microcontroller, or any other suitable device for processing information or instructions. The computing device 150 can be local to the user or can include components that are non-local to the user including one or both of the processor 152 or memory 154 (or portions thereof). For example, in at least some embodiments, the user may operate a terminal that is connected to a non-local computing device. In other embodiments, the memory 154 can be non-local to the user.

The computing device 150 can utilize any suitable processor 152 including one or more hardware processors that may be local to the user or non-local to the user or other components of the computing device. The processor 152 is configured to execute instructions such as instructions provided as part of a demixing engine 155 stored in the memory 154.

Any suitable memory 154 can be used for the computing device 150. The memory 154 illustrates a type of computer-readable media, namely computer-readable storage media. Computer-readable storage media may include, but is not limited to, volatile, nonvolatile, non-transitory, removable, and non-removable media implemented in any method or technology for storage of information, such as computer readable instructions, data structures, program modules, or other data. Examples of computer-readable storage media include RAM, ROM, EEPROM, flash memory, or other memory technology, CD-ROM, digital versatile disks (“DVD”) or other optical storage, magnetic cassettes, magnetic tape, magnetic disk storage or other magnetic storage devices, or any other medium which can be used to store the desired information and which can be accessed by a computing device.

Communication methods provide another type of computer readable media; namely communication media. Communication media typically embodies computer-readable instructions, data structures, program modules, or other data in a modulated data signal such as a carrier wave, data signal, or other transport mechanism and include any information delivery media. The terms “modulated data signal,” and “carrier-wave signal” includes a signal that has one or more of its characteristics set or changed in such a manner as to encode information, instructions, data, and the like, in the signal. By way of example, communication media includes wired media such as twisted pair, coaxial cable, fiber optics, wave guides, and other wired media and wireless media such as acoustic, RF, infrared, and other wireless media.

The display 156 can be any suitable display device, such as a monitor, screen, or the like, and can include a printer. In some embodiments, the display is optional. In some embodiments, the display 156 may be integrated into a single unit with the computing device 150, such as a tablet, smart phone, or smart watch. In at least some embodiments, the display is not local to the user. The input device 158 can be, for example, a keyboard, mouse, touch screen, track ball, joystick, voice recognition system, or any combination thereof, or the like. In at least some embodiments, the input device is not local to the user.

The magnetic field generator(s) 162 can be, for example, Helmholtz coils, solenoid coils, planar coils, saddle coils, electromagnets, permanent magnets, or any other suitable arrangement for generating a magnetic field. As an example, the magnetic field generator 162 can include three orthogonal sets of coils to generate magnetic fields along three orthogonal axes. Other coil arrangement can also be used. The optional sensor(s) 164 can include, but are not limited to, one or more position sensors, orientation sensors, accelerometers, image recorders, or the like or any combination thereof.

The one or more magnetometers 160 can be any suitable magnetometer including, but not limited to, any suitable optically pumped magnetometer. Arrays of magnetometers are described in more detail herein. In at least some embodiments, at least one of the one or more magnetometers (or all of the magnetometers) of the system is arranged for operation in the SERF mode. Examples of magnetic field measurement systems or methods of making such systems or components for such systems are described in U.S. Patent Application Publications Nos. 2020/0072916; 2020/0056263; 2020/0025844; 2020-0057116; 2019/0391213; 2020/0088811; and 2020/0057115; U.S. patent applications Ser. Nos. 16/573,394; 16/573,524; 16/679,048; 16/741,593; and 16/752,393, and U.S. Provisional Patent Applications Ser. Nos. 62/689,696; 62/699,596; 62/719,471; 62/719,475; 62/719,928; 62/723,933; 62/732,327; 62/732,791; 62/741,777; 62/743,343; 62/747,924; 62/745,144; 62/752,067; 62/776,895; 62/781,418; 62/796,958; 62/798,209; 62/798,330; 62/804,539; 62/826,045; 62/827,390; 62/836,421; 62/837,574; 62/837,587; 62/842,818; 62/855,820; 62/858,636; 62/860,001; 62/865,049; 62/873,694; 62/874,887; 62/883,399; 62/883,406; 62/888,858; 62/895,197; 62/896,929; 62/898,461; 62/910,248; 62/913,000; 62/926,032; 62/926,043; 62/933,085; 62/960,548; 62/971,132; and 62/983,406, all of which are incorporated herein by reference in their entireties.

FIG. 1B is a schematic block diagram of one embodiment of a magnetometer 160 which includes a vapor cell 170 (also referred to as a “cell” or “vapor cell”) such as an alkali metal vapor cell; a heating device 176 to heat the cell 170; a pump light source 172a; a probe light source 172b; and a detector 174. In addition, coils of a magnetic field generator 162 can be positioned around the vapor cell 170. The vapor cell 170 can include, for example, an alkali metal vapor (for example, rubidium in natural abundance, isotopically enriched rubidium, potassium, or cesium, or any other suitable alkali metal such as lithium, sodium, or francium) and, optionally, one, or both, of a quenching gas (for example, nitrogen) and a buffer gas (for example, nitrogen, helium, neon, or argon). In some embodiments, the vapor cell may include the alkali metal atoms in a prevaporized form prior to heating to generate the vapor.

The pump and probe light sources 172a, 172b can each include, for example, a laser to, respectively, optically pump the alkali metal atoms and probe the vapor cell. The pump and probe light sources 172a, 172b may also include optics (such as lenses, waveplates, collimators, polarizers, and objects with reflective surfaces) for beam shaping and polarization control and for directing the light from the light source to the cell and detector. Examples of suitable light sources include, but are not limited to, a diode laser (such as a vertical-cavity surface-emitting laser (VCSEL), distributed Bragg reflector laser (DBR), or distributed feedback laser (DFB)), light-emitting diode (LED), lamp, or any other suitable light source.

The detector 174 can include, for example, an optical detector to measure the optical properties of the transmitted probe light field amplitude, phase, or polarization, as quantified through optical absorption and dispersion curves, spectrum, or polarization or the like or any combination thereof. Examples of suitable detectors include, but are not limited to, a photodiode, charge coupled device (CCD) array, CMOS array, camera, photodiode array, single photon avalanche diode (SPAD) array, avalanche photodiode (APD) array, or any other suitable optical sensor array that can measure the change in transmitted light at the optical wavelengths of interest.

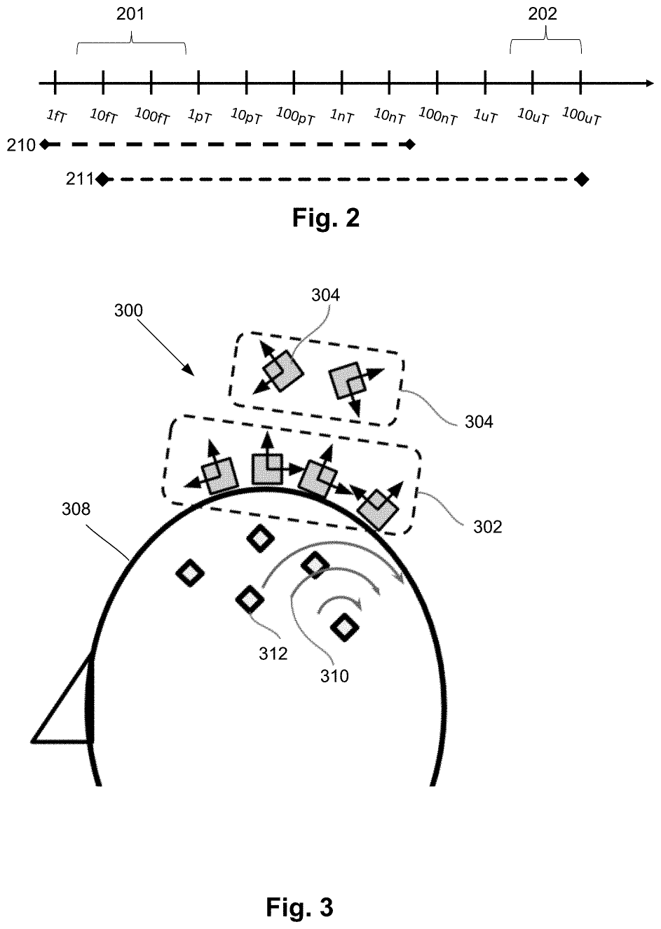

FIG. 2 shows the magnetic spectrum from 1 fT to 100 μT in magnetic field strength on a logarithmic scale. The magnitude of magnetic fields generated by the human brain are indicated by range 201 and the magnitude of the background ambient magnetic field, including the Earth's magnetic field, by range 202. The strength of the Earth's magnetic field covers a range as it depends on the position on the Earth as well as the materials of the surrounding environment where the magnetic field is measured. Range 210 indicates the approximate measurement range of a magnetometer (e.g., an OPM) operating in the SERF mode (e.g., a SERF magnetometer) and range 211 indicates the approximate measurement range of a magnetometer operating in a scalar mode (e.g., a scalar magnetometer.) Typically, a SERF magnetometer is more sensitive than a scalar magnetometer but many conventional SERF magnetometers typically only operate up to about 0 to 200 nT while the scalar magnetometer starts in the 10 to 100 fT range but extends above 10 to 100 μT.

In both shielded and unshielded environments, the magnetic fields detected by a magnetic field measurement system, such as a magnetoencephalography (MEG) system, are a mixture of magnetic fields for measurement (for example, magnetic fields originating from one or more magnetic field sources of interest such as a neural source in the brain or elsewhere) and the ambient background magnetic field (arising from the environment) or other magnetic fields that are not of interest (for example, non-neural physiological magnetic fields.) It is often desirable to de-mix these detected signals at an early stage of processing to remove any confounds caused by mixed measurement (e.g., from the magnetic field source(s) of interest) and background components of the magnetic field signals. Many, if not all, existing conventional systems and methods for performing this de-mixing rely heavily on precise knowledge of the locations, orientations, and calibrations of the magnetic field sensors (e.g., OPMs) relative to each other. Moreover, existing conventional noise suppression techniques often have requirements for calibration precision and can be computationally complex. In some MEG systems, consumer grade systems for example, it may be infeasible to precisely know the relative locations, orientations, or calibrations of sensors. This is particularly true with a modular MEG system, where groups of sensors can be placed independently.

The systems and methods described herein utilize a physical arrangement that includes a number of magnetic field sensors (for example, magnetometers such as OPMs) oriented and positioned in a particular configuration and a relatively computationally simple software system that allows for time-varying de-mixing of neural and non-neural signals given some knowledge of the positions, orientations, or calibrations of the magnetic field sensors (for example, a grouping based on distance from a user's scalp).

The systems and methods described herein will be exemplified using the measurement of magnetic fields generated by neural tissue in the brain of a user.

The systems and methods described herein utilize magnetic field sensors (also termed “sensors”) which can be magnetometers such as OPMs. In at least some embodiments, other magnetic field sensors may be used in addition to, or as an alternative to, OPMs.

FIG. 3 illustrates one embodiment of a magnetic field measurement system 300 that includes a first group of magnetic field sensors 302 and a second group of magnetic field sensors 304 that are positioned relative to one or more magnetic field sources 312 of interest. The magnetic field measurement system 300 can be in a shielded environment, such as a shielded room, or in an unshielded environment.

All of the magnetic field sensors 302, 304 are mounted relative to the magnetic field source 312 of interest (or the user's head 308) and, preferably, maintain the same position relative to each other. In at least some embodiments, each magnetic field sensor 302, 304 is configured to be sensitive to one or more magnetic field orientations, as shown by the arrows emanating from the magnetic field sensors 302, 304 in FIG. 3.

The magnetic field sensors 302, 304 of the first and second groups are arranged in any suitable configuration and, preferably, are disposed in a single article or set of joined articles. As an example, the first and second groups of magnetic field sensors 302, 304 can be disposed in a wearable article, such as a helmet, hat, beanie, hood, cap, scarf, or the like that can be placed on the head 308 of a user. Examples of wearable conformable MEG systems that would cover part of the user's head are described in U.S. Non-Provisional patent application Ser. No. 16/457,655 which is incorporated herein by reference in its entirety.

In at least some embodiments, the magnetic field sensors 302, 304 are categorized into two groups: 1) the first group of magnetic field sensors or target sensors 302 (or “first magnetic field sensors”) and 2) the second group of magnetic field sensors or external sensors 304 (or “second magnetic field sensors”). The target sensors 302 are positioned and oriented in a way that allows for these target sensors to be sensitive to a magnetic field 310 emanating from one or more target sources 312 of interest (which are also referred to herein as “magnetic field sources of interest”). In at least some embodiments, the target sensors 302 (or first magnetic field sensors) and the external sensors 304 (or second magnetic field sensors) are configured and arranged so that the target sensors can be positioned to receive signals from the target source(s) with the target sensors 302 positioned closer to the target source(s) than the second magnetic field sensors 304. In at least some embodiments, the collection of magnetic field sensors 302, 304 are arranged in relatively close proximity to the target source 312 that is to be detected (for example, within 15 cm or less from the user's head 308 for detection of magnetic fields generated in the brain.)

As an example, in FIG. 3, the target sensors 302 are positioned and oriented to monitor a magnetic field 310 originating from one or more neural sources (i.e., one or more target sources 312) inside of the user's head 308. In at least some embodiments of a MFG system, the target sensors 302 are positioned very close (for example, 2 cm or less) to the surface of the scalp. The target sensors 302 and external sensors 304 can be disposed in a wearable article, such as a helmet, hat, hood, cap, scarf, or the like that can he placed on the head 308 of a user

In at least some embodiments, the target sensors 302 may also be positioned and oriented such that the target sensors 302 share little or no information with each other regarding the magnetic field(s) 310 from the one or more target sources 312. example, the target sensors 302 can be located relatively far (for example, at least 4 cm) from each other or, as illustrated in FIG. 3, the target sensors 302 can be located near each other (for example, 4 cm or less distant), but have different (for example, orthogonal) orientations. These target sensors 302 will also be sensitive to the ambient background magnetic field (e.g., the magnetic field generated from sources other than the target source(s)) which includes magnetic fields originating from external sources, such as the Earth, electronic devices, or the like.

The external sensors 304 are positioned and oriented such that they have little or no sensitivity to magnetic field(s) 310 originating from the one or more target sources 312 (for example, the neural magnetic field sources inside of the user's head 308.) These external sensors 304, however, are individually positioned and oriented such that the external sensors 304 are sensitive to the ambient background magnetic field (which arises from other (or external) sources—i.e., non-target sources) to which some set of target sensors 302 are also sensitive. In at least some embodiments, the positions and orientations of the external sensors 304 may be selected so that each external sensor shares little or no signal sensitivity with other external sensors (for example, the external sensors may have orthogonal orientations), as this may provide for better target/external (e.g., non-target) signal separation using fewer external sensors.

The measured signals (e.g., the multi-channel signals) from the magnetic field sensors 302, 304 can be processed or recorded by a computer system (for example, computing device 150 of FIG. 1A), either in real-time or offline. The measured signals can be transformed or demixed by the computer system into separate signals from a) the target source(s) 312 and b) the magnetic field(s) arising from other (or external) sources (e.g., the ambient background magnetic field).

In at least some embodiments, the only information needed about the magnetic field sensors from which the measured signals come is to which set each of the magnetic field sensors belongs: either the set of target sensors 302 or the set of external sensors 304. An underlying assumption is that the target sensors 302 will be sensitive to a linear combination of fields from both target and external sources, while the external sensors will be sensitive to a linear combination of fields from only external sources. Although some magnetic field(s) 310 from one or more target sources 312 may reach the external sensors 304, it is assumed that the fields are sufficiently small as to be ignored. Another aspect of this assumption is that the external sources are far enough away (for example, at least 1 meter distant so that the distance between the external sensors 304 and target sensors 302 can be considered small relative to the distance from the external source) such that the measured fields (from external sources) at the sensors 302, 304 behave linearly.

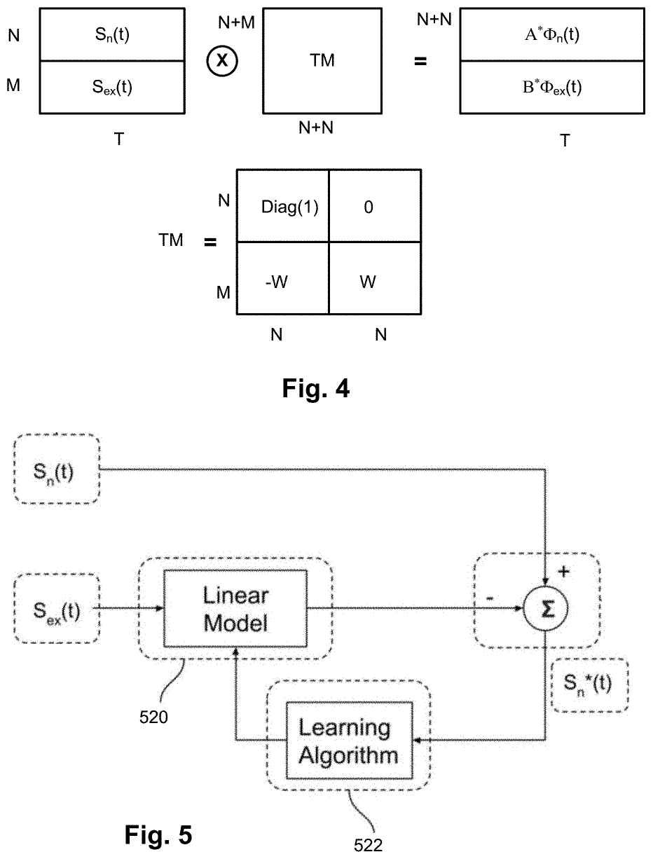

The demixing engine 155 of the computing device 150 (or any other computing device) can be used to separate the signals from the one or more target sources 312 and the signals from the other (or external) sources. It will be understood that the demixing engine 155 may be distributed over multiple processors or computing devices. FIGS. 4 and 5 illustrate aspects of at least some embodiments of the demixing engine 155.

FIG. 4 illustrates calculational elements for processing sensor signals including the measured signal matrix Sₙ(t) from the N target sensors 302 (FIG. 3), the measured signal matrix Sₑₓ(t) from the M external sensors 304, the (N+M)×(N+N) transformation matrix TM, the estimate of the signal from the target sources in sensor space A*Φₙ(t), and the estimate of the signal from the external sources in sensor space B*Φₑₓ(t). The transformation matrix takes the form illustrated in FIG. 4 where W is defined below.

The target sensor measurements Sₙ(t) and external sensor measurements Sₑₓ(t) can be written as the following:

Sₙ(t)=A*Φₙ(t)+B*Φₑₓ(t)+εₙ(t) 1)

where:

Sₙ(t) is the measured signal matrix from the target sensors 302;

Φₙ(t) is the matrix of magnetic fields from all target sources;

Φₑₓ(t) is the matrix of magnetic fields from all external sources; and

εₙ(t) is the neural measurement noise matrix; and.

Sₑₓ(t)=CΦₑₓ(t)+εₑₓ(t) 2)

where:

Sₑₓ(t) is the measured signal matrix from the external sensors 304; and

εₑₓ(t) is the external measurement noise matrix.

In both equations above, A*, B*, and C, are forward matrices that map target and external magnetic fields to target and external sensors.

Using an inverse model of Equation 2, Φₑₓ(t)=C′Sₑₓ(t)+ε′ₑₓ(t), equation (1) is rewritten as:

Sₙ(t)=A*Φₙ(t)+B*C′Sₑₓ(t)+ε″(t) 3)

and defining B*C′ [symbol] W, results in:

Sₙ(t)−WSₑₓ(t)=A*Φₙ(t)+ε″(t) 4)

which indicates that a linear transformation of measurement signal(s) from the external sources, subtracted from the measurement signal(s) from the target sources, extracts the target source component of the overall measurement(s). Note that all noise terms have been combined into ε″(t).

To find W, least squares (or any other appropriate method) can be used, treating A*Φₙ(t)+ε″(t) as an uncorrelated error term. More specifically, Sₑₓ(t) is a regressor and Sₙ(t) is a target, to find W* that minimizes the following:

$$5 ) {W}^{*} = arg {min}_{W ∈ {ℛ}^{M \times N}} {‖ {S}_{n} ( t ) - {WS}_{e x} ( t ) ‖}_{2}$$

Which in turn gives

S*ₙ(t)=Sₙ(t)−W*Sₑₓ(t) 6)

where S*ₙ(t) is the matrix of target sensor measurement signal(s) with an estimate of the portion of the signal arising from the external sources (e.g., the ambient background magnetic field and other sources) removed, N is the number of measured signals from the target sensors, and M is the number of measured signals from external sensors. Again, the assumption is that Φₙ(t) and Sₑₓ(t) are independent, thus this subtraction should not remove the signal(s) from the target source(s).

In at least some embodiments, estimation is sufficient given a sufficient number of samples (for example, in at least some embodiments, sampling over no more than 120 seconds based on initial testing). In at least some embodiments, the transformation has been found to be stable over at least 20 minutes in a shielded room.

In at least some embodiments, the measured signals may be temporally filtered (for example, bandpass filtered) before the above steps to avoid overfitting of certain noise sources or DC components.

The weights W* can be updated with time. As one example, a long (for example, at least 30 s) moving window of measurements can be used to calculate W* and update equation 6 at selected intervals. As another example, the weights W* can be updated when the noise in the transformed signals given by equation 6 crosses a certain threshold indicating that the update of W* may be helpful.

Upon completion of the transformation, the transformed signals then can be further processed as if they were in sensor space.

Equations 5 and 6 can be generalized so that the projection can take any form. In the most general form, equations 5 and 6 are written as the following:

$$7 ) {F}^{*} = arg {min}_{F ∈ ℱ} {‖ {S}_{n} - F ( {S}_{e x} ) ‖}_{2} \\ 8 ) {S}_{n}^{*} = {S}_{n} - {F}^{*} ( {S}_{ex} )$$

Where the function F can be nonlinear and can have memory. [symbol] is the space of all variations of F.

Another embodiment utilizes a spatio-temporal linear model. The linear model described above does not have any temporal component to it. In other words, the system in equations 5 and 6 is memory-less. This condition is relaxed by considering models that have a temporal component as well, so that equation 5 can be rewritten as:

$$9 ) {W}^{*} = arg {min}_{W ∈ {ℛ}^{M \times N \times k}} {‖ {S}_{n} ( t ) - {\sum }_{τ = 0}^{k - 1} W ( τ ) {S}_{e x} ( τ ) ‖}_{2}$$

and equation 6 becomes:

S*ₙ(t)=Sₙ(t)−Σ_{τ=0}^{k-1}W*(τ)Sₑₓ(t−τ) 10)

where k is the number of time delays for the spatiotemporal linear model. In this case, W* is a three-dimensional matrix instead of two dimensional.

One embodiment of an arrangement utilizing this spatio-temporal linear model is illustrated in FIG. 5. Background sensor measurements Sₑₓ(t) are input into a linear model 520 which is given by the weights W* derived from equation 9. The result is subtracted from the measurements Sₙ(t) as illustrated in FIG. 5. The result of this subtraction is the cleaned neural signal S*ₙ(t).

The preceding embodiment described above uses a linear model for function F (equation 7). In a more generalized case, this function can be nonlinear. An example of such nonlinear functions are neural networks. In some embodiments, the linear model 520 in FIG. 5 can be replaced with a nonlinear function, such as a neural network model. In the case of a neural network mode, different algorithms can used to find W*. Examples include, but are not limited to, stochastic gradient descent, adaptive gradient, adaptive gradient with momentum, and Gauss-Newton method.

As illustrated in FIG. 5, the cleaned neural signal S*ₙ(t) can also serve as the error term for a learning algorithm 522 to adjust the weights of the linear system 520. For example, a learning algorithm can monitor the difference between Sₙ(t) and S*ₙ(t) and update the linear model if this difference exceeds a predetermined threshold. Any suitable learning algorithm can be used. One example of a suitable learning algorithm utilizes the elastic net regression method

Sensor weighting is also a consideration. In at least some embodiments, weighting of the external sensors can be provided according to distance from each target sensor. In some embodiments, external sensors closer to a target sensor are weighted more heavily so that more of the signal from the external sources is removed. In some embodiments, external sensors further from a target sensor are weighted more heavily so that less activity from the target source(s) is removed. In some embodiments, sensors that are overly noisy or otherwise giving bad measurements can be weighted less or excluded.

The systems and methods described herein can include one or more of the following features. In at least some embodiments, external or environmental reference sensors are mounted to the head along with target sensors as described above. In at least some embodiments, the system or method may utilize only knowledge of external versus target sensor groups. In at least some embodiments, the system or method may utilize relatively simple linear regression methods for head worn MEG. In at least some embodiments, weights can be updated in real-time using a long moving window. In at least some embodiments, the system or method may utilize an adaptive filter method for head worn MEG. In at least some embodiments, the system or method may utilize a neural network method for head worn MEG.

The methods described herein can be implemented in the demixing engine 155. It will be understood that components or functions of the demixing engine 155 can be present in a single device or can be distributed among multiple devices that can be connected through a wired or wireless network.

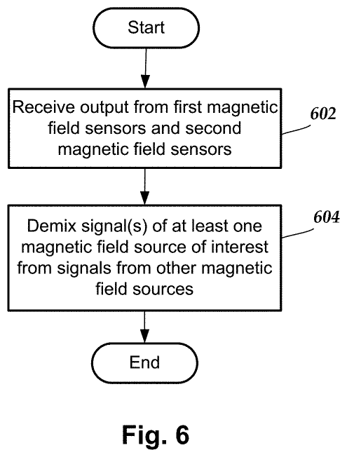

FIG. 6 illustrates one embodiment of a method of demixing signal(s) from one or more magnetic field sources of interest from signals from other magnetic field sources. In step 602, output is received from first magnetic field sensors and from second magnetic field sensors. The first and second magnetic field sensors are positioned so that the first magnetic field sensors positioned closer to the at least one target source than the second magnetic field sensors.

In step 604, using the output of the first and second magnetic field sensors, signal(s) from the at least one target source is demixed from signals from other magnetic field sources. The demixing can be performed using any of the models described above including, but not limited to, the linear models, non-linear models, spatio-temporal linear models, and spatio-temporal non-linear models described above. The demixed signal from the at least one target source of interested can then be used for a variety of applications including, but not limited to, identifying the neural origin of the demixed signal, extracting information from the demixed signal that is correlated with a certain brain function such as working memory or vision.

In at least some embodiments, the systems or methods can include one or more of the following advantages: a computationally simple method to find transformation which can be updated frequently; a method with simple matrix multiplication to apply transformation; a method that uses a relatively small amount of knowledge of sensor positions, orientations, or gain calibrations (for example, the method may only use knowledge of whether a sensor is external or neural); a system or method in which, after cleaning/filtering (for example, bandpass filtering the measured MEG signals), the resulting neural signals may still be treated as if they are in the original sensor space,

Examples of magnetic field measurement systems in which the embodiments presented above can be incorporated, and which present features that can be incorporated in the embodiments presented herein, are described in U.S. Patent Application Publications Nos. 2020/0072916; 2020/0056263; 2020/0025844; 2020-0057116; 2019/0391213; 2020/0088811; and 2020/0057115; U.S. patent application Ser. Nos. 16/573,394; 16/573,524; 16/679,048; 16/741,593; and 16/752,393, and U.S. Provisional Patent Applications Ser. Nos. 62/689,696; 62/699,596; 62/719,471; 62/719,475; 62/719,928; 62/723,933; 62/732,327; 62/732,791; 62/741,777; 62/743,343; 62/747,924; 62/745,144; 62/752,067; 62/776,895; 62/781,418; 62/796,958; 62/798,209; 62/798,330; 62/804,539; 62/826,045; 62/827,390; 62/836,421; 62/837,574; 62/837,587; 62/842,818; 62/855,820; 62/858,636; 62/860,001; 62/865,049; 62/873,694; 62/874,887; 62/883,399; 62/883,406; 62/888,858; 62/895,197; 62/896,929; 62/898,461; 62/910,248; 62/913,000; 62/926,032; 62/926,043; 62/933,085; 62/960,548; 62/971,132; and 62/983,406, all of which are incorporated herein by reference.

The methods, systems, and units described herein may be embodied in many different forms and should not be construed as limited to the embodiments set forth herein. Accordingly, the methods, systems, and units described herein may take the form of an entirely hardware embodiment, an entirely software embodiment or an embodiment combining software and hardware aspects. The methods described herein can be performed using any type of processor or any combination of processors where each processor performs at least part of the process.

It will be understood that each block of the flowchart illustrations, and combinations of blocks in the flowchart illustrations and methods disclosed herein, can be implemented by computer program instructions. These program instructions may be provided to a processor to produce a machine, such that the instructions, which execute on the processor, create means for implementing the actions specified in the flowchart block or blocks disclosed herein. The computer program instructions may be executed by a processor to cause a series of operational steps to be performed by the processor to produce a computer implemented process. The computer program instructions may also cause at least some of the operational steps to be performed in parallel. Moreover, some of the steps may also be performed across more than one processor, such as might arise in a multi-processor computer system. In addition, one or more processes may also be performed concurrently with other processes, or even in a different sequence than illustrated without departing from the scope or spirit of the invention.

The computer program instructions can be stored on any suitable computer-readable medium including, but not limited to, RAM, ROM, EEPROM, flash memory or other memory technology, CD-ROM, digital versatile disks (“DVD”) or other optical storage, magnetic cassettes, magnetic tape, magnetic disk storage or other magnetic storage devices, or any other medium which can be used to store the desired information and which can be accessed by a computing device.

The above specification provides a description of the invention and its manufacture and use. Since many embodiments of the invention can be made without departing from the spirit and scope of the invention, the invention also resides in the claims hereinafter appended.

What is claimed as new and desired to be protected by Letters Patent of the United States is:

1. A magnetic field measurement system, comprising:
a plurality of first magnetic field sensors and a plurality of second magnetic field sensors, wherein the first and second magnetic field sensors are configured and arranged so that the first magnetic field sensors are positionable to receive at least one signal from at least target source with the first magnetic field sensors positioned closer to the at least one target source than the second magnetic field sensors;
at least one memory;
at least one processor coupled to the at least one memory and the first and second magnetic field sensors and configured to receive output of the first and second magnetic field sensors, wherein the at least one processor is configured to perform actions comprising;
receiving output of the first and second magnetic field sensors; and
demixing, using the output of the first and second magnetic field sensors, the at least one signal from the at least one target source from signals from other magnetic field sources.

2. The magnetic field measurement system of claim 1, wherein the demixing is performed using a model in which the output of the first magnetic field sensors comprises the at least one signal from the at least one target source and that the output of the second magnetic field sensors does not comprise the at least one signal from the at least one target source.

3. The magnetic field measurement system of claim 2, wherein the demixing utilizes a linear model of the signal from the at least one target source and the other magnetic field sources.

4. The magnetic field measurement system of claim 3, wherein the linear model comprises the following equations:
Sₙ(t)=A*Φₙ(t)+B*Φₑₓ(t)+εₙ(t)
Sₑₓ(t)=CΦₑₓ(t)+εₑₓ(t)
wherein
Sₙ(t) is a measured signal matrix from the first magnetic field sensors;
Φₙ(t) is a matrix of fields from the at least one target source;
Φₑₓ(t) is a matrix of fields from the other magnetic field sources;
εₙ(t) is a first measurement noise matrix;
Sₑₓ(t) is a measured signal matrix from the second magnetic field sensors;
εₑₓ(t) is a second measurement noise matrix;
A is a matrix that maps the at least one target source to the first magnetic field sensors;
B is a matrix that maps the other magnetic field sources to the first magnetic field sensors; and
C is a matrix that maps the other magnetic field sources to the second magnetic field sensors.

5. The magnetic field measurement system of claim 4, wherein the demixing further comprises finding W, a M×N matrix from the space [symbol] ^{M×N}, that minimizes the following:
$${W}^{*} = arg {min}_{W ∈ {ℛ}^{M \times N}} {‖ {S}_{n} ( t ) - {WS}_{e x} ( t ) ‖}_{2}$$
to give
S*ₙ(t)=Sₙ(t)−W*Sₑₓ(t)
wherein
S*ₙ(t) is a signal matrix from the first magnetic field sensors with an estimate of the signals from the other magnetic field sources removed;
N is the number of first magnetic field sensors; and
M is the number of second magnetic field sensors.

6. The magnetic field measurement system of claim 5, wherein the actions further comprise:
adjusting W by applying S*ₙ(t) as an error term to a learning algorithm.

7. The magnetic field measurement system of claim 4, wherein the demixing further comprises finding time-varying W(t), a M×N×k matrix from the space [symbol] ^{M×N×k}, that minimizes the following:
$${W}^{*} = arg {min}_{W ∈ {ℛ}^{M \times N \times k}} {‖ {S}_{n} ( t ) - {\sum }_{τ = 0}^{k - 1} W ( τ ) {S}_{e x} ( τ ) ‖}_{2}$$
to give
S*ₙ(t)=Sₙ(t)−Σ_{τ=0}^{k-1}W*(τ)Sₑₓ(t−τ)
wherein
S*ₙ(t) is a signal matrix from the first magnetic field sensors with an estimate of the signals from e other magnetic field sources removed;
N is the number of first magnetic field sensors;
M is the number of second magnetic field sensors; and
k is a number of time increments.

8. The magnetic field measurement system of claim 2, wherein the demixing utilizes a non-linear model of the signals from the at least one target source and the other magnetic field sources.

9. The magnetic field measurement system of claim 8, wherein the non-linear model comprises the following equations:
Sₙ(t)=A*Φₙ(t)+B*Φₑₓ(t)+εₙ(t)
Sₑₓ(t)=CΦₑₓ(t)+εₑₓ(t)
wherein
Sₙ(t) is a measured signal matrix from the first magnetic field sensors;
Φₙ(t) is a matrix of fields from the at least one target source;
Φₑₓ(t) is a matrix of fields from the other magnetic field sources;
εₙ(t) is a first measurement noise matrix;
Sₑₓ(t) is a measured signal matrix from the second magnetic field sensors;
εₑₓ(t) is a second measurement noise matrix;
A is a matrix that maps the at least one target source to the first magnetic field sensors;
B is a matrix that maps the other magnetic field sources to the first magnetic field sensors; and
C is a matrix that maps the other magnetic field sources to the second magnetic field sensors.

10. The magnetic field measurement system of claim 9, wherein the demixing further comprises finding F, a non-linear function from the space [symbol] , that minimizes the following:
$${F}^{*} = arg {min}_{F ∈ ℱ} {‖ {S}_{n} - F ( {S}_{e x} ) ‖}_{2}$$
to give
S*ₙ=Sₙ−F*(Sₑₓ)
wherein
S*ₙ(t) is a signal matrix from the first magnetic field sensors with an estimate of the signals from the other magnetic field sources removed.

11. The magnetic field measurement system of claim 10, wherein the actions further comprise:
adjusting F by applying S*ₙ(t) as an error term to a learning algorithm.

12. The magnetic field measurement system of claim 1, wherein the first and second magnetic field sensors are disposed in a wearable article configured for placement on a head of a user.

13. The magnetic field measurement system of claim 12, wherein, when the wearable article is placed on the head of the user, the first magnetic field sensors are positioned closer to the head of the user than the second magnetic field sensors.

14. A non-transitory computer-readable medium having stored thereon instructions for execution by a processor, including:
receiving output of a plurality of first magnetic field sensors and a plurality of second magnetic field sensors; and
demixing, using the output of the first and second magnetic field sensors, at least one signal from at least one target source from signals from other magnetic field sources, wherein the demixing is performed using a model in which the output of the first magnetic field sensors comprises the at least one signal from the at least one target source and that the output of the second magnetic field sensors does not comprise the at least one signal from the at least one target source.

15. The non-transitory computer-readable medium of claim 14, wherein the demixing utilizes a linear model of the signals from the at least one target source and the other magnetic field sources.

16. The non-transitory computer-readable medium of claim 15, wherein the linear model comprises the following equations:
Sₙ(t)=A*Φₙ(t)+B*Φₑₓ(t)+εₙ(t)
Sₑₓ(t)=CΦₑₓ(t)+εₑₓ(t)
wherein
Sₙ(t) is a measured signal matrix from the first magnetic field sensors;
Φₙ(t) is a matrix of fields from the at least one target source;
Φₑₓ(t) is a matrix of fields from the other magnetic field sources;
εₙ(t) is a first measurement noise matrix;
Sₑₓ(t) is a measured signal matrix from the second magnetic field sensors;
εₑₓ(t) is a second measurement noise matrix;
A is a matrix that maps the at least one target source to the first magnetic field sensors;
B is a matrix that maps the other magnetic field sources to the first magnetic field sensors; and
C is a matrix that maps the other magnetic field sources to the second magnetic field sensors.

17. The non-transitory computer-readable medium of claim 16, wherein the demixing further comprises finding W, a M×N matrix from the space [symbol] ^{M×N}, that minimizes the following:
$${W}^{*} = arg {min}_{W ∈ {ℛ}^{M \times N}} {‖ {S}_{n} ( t ) - {WS}_{e x} ( t ) ‖}_{2}$$
to give
S*ₙ(t)=Sₙ(t)−W*Sₑₓ(t)
wherein
S*ₙ(t) is a signal matrix from the first magnetic field sensors with an estimate of the signals from the other magnetic field sources removed;
N is the number of first magnetic field sensors; and
M is the number of second magnetic field sensors.

18. The non-transitory computer-readable medium of claim 16, wherein the demixing further comprises finding time-varying W(t), a M×N×k matrix from the space [symbol] ^{M×N×k}, that minimizes the following:
$${W}^{*} = arg {min}_{W ∈ {ℛ}^{M \times N \times k}} {‖ {S}_{n} ( t ) - {\sum }_{τ = 0}^{k - 1} W ( τ ) {S}_{e x} ( τ ) ‖}_{2}$$
to give
S*ₙ(t)=Sₙ(t)−Σ_{τ=0}^{k-1}W*(τ)Sₑₓ(t−τ)
wherein
S*ₙ(t) is a signal matrix from the first magnetic field sensors with an estimate of the signals from the other magnetic field sources removed;
N is the number of first magnetic field sensors;
M is the number of second magnetic field sensors; and
k is a number of time increments.

19. The non-transitory computer-readable medium of claim 14, wherein the demixing utilizes a non-linear model of the signals from the at least one target source and the other magnetic field sources.

20. The non-transitory computer-readable medium of claim 19, wherein the model comprises the following equations:
Sₙ(t)=A*Φₙ(t)+B*Φₑₓ(t)+εₙ(t)
Sₑₓ(t)=CΦₑₓ(t)+εₑₓ(t)
wherein
Sₙ(t) is a measured signal matrix from the first magnetic field sensors;
Φₙ(t) is a matrix of fields from the at least one target source;
Φₑₓ(t) is a matrix of fields from the other magnetic field sources;
εₙ(t) is a first measurement noise matrix;
Sₑₓ(t) is a measured signal matrix from the second magnetic field sensors;
εₑₓ(t) is a second measurement noise matrix;
A is a matrix that maps the at least one target source to the first magnetic field sensors;
B is a matrix that maps the other magnetic field sources to the first magnetic field sensors; and
C is a matrix that maps the other magnetic field sources to the second magnetic field sensors.

21. The non-transitory computer-readable medium of claim 20, wherein the demixing further comprises finding F, a non-linear function from the space [symbol] , that minimizes the following:
$${F}^{*} = arg {min}_{F ∈ ℱ} {‖ {S}_{n} - F ( {S}_{e x} ) ‖}_{2}$$
to give
S*ₙ=Sₙ−F*(Sₑₓ)
wherein
S*ₙ(t) is a signal matrix from the first magnetic field sensors with an estimate of the signals from the other magnetic field sources removed.
